(19) United States
(12) Patent Application Publication
(10) Pub. No.: US 2024/0398210 A1
(43) Pub. Date: Dec. 5, 2024

(54) ENDOSCOPE HANDLE AND ENDOSCOPE WITH HANDLE

(71) Applicant: AMBU A/S, Ballerup (DK)

(72) Inventors: Kaspar Mat MATTHISON-HANSEN, Helsingør (DK); Tue Kjærgaard TOFT, Kongens Lyngby (DK)

(73) Assignee: AMBU A/S, Ballerup (DK)

(21) Appl. No.: 18/698,520

(22) PCT Filed: Oct. 18, 2022
(86) PCT No.: PCT/EP2022/078943

§ 371 Date: Apr. 4, 2024

(30) Foreign Application Priority Data
Oct. 19, 2021 (DK) PA 2021 70514

(51) Int. Cl. A61B 1/00

(52) U.S. Cl. CPC A61B 1/00066

(57) ABSTRACT

A control handle for an endoscope and an endoscope having the control handle, the control handle including a handle housing having a proximal end, a distal end longitudinally opposite the proximal end, and a grip section between the proximal end and the distal end, the grip section having a palm surface, a finger surface opposite the palm surface, and elongate longitudinal recesses intermediate the finger surface and the palm surface, the palm surface being convex, and the finger surface being convex.

### CROSS-REFERENCE TO RELATED APPLICATIONS

None.

### TECHNICAL FIELD

The present disclosure relates to endoscopes and endoscope handles, in particular single-use endoscopes.

### BACKGROUND

Endoscopes are typically equipped with a light source and a vision receptor including a vision or image sensor. Provided that enough light is present, it is possible for an operator to see where the endoscope is steered and to set the target of interest once the tip has been advanced thereto.

Endoscopes encompass a broad category of devices operable to obtain and present images with a display screen. Endoscopes include, generally, a handle, an insertion tube extending from the handle, a bending section distally of the insertion tube, and a camera distally of the bending section. The combination of the insertion tube, the bending section, and a distal tip comprising a camera may be referred to as a “cord”. The camera generally includes an image sensor and a light source. The handle includes bending controls operable to articulate the bending section and thereby orient the field of view of the camera. Endoscopes include, for example, bronchoscopes, cholangioscopes, colonoscopes, ear-nose-throat scopes, duodenoscopes, ureteroscope, and other medical devices having an image sensor at the distal end of an insertion tube and configured to obtain images of internal tissue of a patient, for example a cord with an ultrasound sensor to combine endoscopy with ultrasound imaging. Medical monitors and video processing apparatus can be communicatively coupled to the endoscopes to receive image data therefrom and present images corresponding to the image data on a display screen. Endoscopes may include various additional controls and features.

An endoscope described in commonly owned U.S. Patent Application No. 2019/0223694 has an insertion tube with an internal working channel and a connector at the handle adapted for the attachment of a syringe. A recess is adapted to accommodate a cylindrical body of the syringe when the syringe is attached to the connector. The endoscope is adapted to perform bronchoalveolar lavage, a procedure for obtaining samples, through the working channel, of organic material from a lung segment of a patient. Commonly owned U.S. Pat. No. 10,321,804, incorporated by reference herein, describes an articulated tip of an endoscope. Commonly owned U.S. Pat. No. 9,220,400 describes a camera housing arranged at the distal end of the insertion tube. The camera housing is molded and contains an image sensor and a light source, e.g. LED, embedded in the material of the camera housing. The foregoing application and patents describe technical characteristics of respective endoscopes described therein and are incorporated herein by reference in their entirety.

The bending section may be bendable in one or two dimensions, e.g. along a palm/finger dimension and a left/right dimension. The endoscope is articulated by tensioning or slacking steering wires running along the inside of the insertion tube from the tip thereof through articulated segments of the bending section to a control system or control mechanism positioned in or forming part of the handle. An endoscope control system with a two-dimensional bending control system is disclosed in U.S. Pat. No. 10,357,634, which is incorporated by reference herein.

Endoscopes may also include control elements, such as buttons, positioned in the handle and operable to control operation of the camera, monitor, video processing apparatus, etc. As endoscopes increase in complexity it becomes increasingly more difficult to provide such control elements in a manner that permits the operator to grasp and operate the control elements to control the endoscope with one hand.

### SUMMARY

A first aspect of this disclosure relates to a control handle for an endoscope, the control handle including a handle housing having a proximal end, a distal end longitudinally opposite the proximal end, and a grip section between the proximal end and the distal end, the grip section having a palm surface, a finger surface opposite the palm surface, and elongate longitudinal recesses intermediate the finger surface and the palm surface, the palm surface being convex, and the finger surface being convex.

Each of the elongate longitudinal recesses have an elongate recess surface between the palm surface and the finger surface, a profile of the elongate recess surface in the transverse plane being different than a profile of the palm surface and a profile of the finger surface. The profile of the elongate recess surface, along the transverse plane, is flatter than the profiles of the finger surface and the palm surface, or flat or concave.

The profile of the elongate recess surface, along the transverse plane, is such that the elongate recess surface does not intersect a line, along the transverse plane, extending from the palm surface to the finger surface.

In some embodiments, the grip section is symmetric about a longitudinal plane E-E extending therethrough.

In some embodiments, the finger surface includes a longitudinal convex surface and an undercut surface connected to and extending proximally from the longitudinal convex surface.

The undercut surface may be curved along the longitudinal plane and curve toward the palm surface, a portion of the undercut surface positioned between a longitudinal projection of the longitudinal convex surface and the palm surface.

In some embodiments, the undercut surface is omitted.

In some embodiments, the profile of the elongate recess surface in the transverse plane is substantially flat along the grip section. By substantially flat it is meant that a radius of curvature is at least 3, 4, 5, 6, 7, or 8 times greater than the radius of curvature of the palm surface.

In some embodiments, the elongate recess surface extends along the entire grip section.

In some embodiments, a length of the elongate recess surface defines a length of the grip section.

In some embodiments, the profile of the elongate recess surface in the transverse plane is substantially concave.

In some embodiments, the grip section is symmetric about a longitudinal plane extending therethrough.

The grip section may exhibit a cone shape.

The palm surface may define a line “a” on the longitudinal plane, the finger surface may define a line “b” on the longitudinal plane, and the lines a and b may form an angle with a vertex distal of the distal end of the handle housing. The lines may be straight or slightly curved such that the cone shape can be envisioned.

The palm surface may define a straight line “a” on the longitudinal plane, the finger surface may define a straight line “b” on the longitudinal plane, and the straight lines a and b may form an angle with a vertex distal of the distal end of the handle housing.

In some embodiments, the control handle housing comprises a palm side and a finger side opposite the palm side, a suction button at the proximal end on the finger side of the handle housing; and a bending control lever at the proximal end on the palm side of the handle housing.

In some embodiments, the palm surface and/or the finger surface are textured.

A second aspect of the present disclosure relates to an endoscope comprising an endoscope handle according to the first aspect.

The endoscope may further comprise an insertion tube at a distal end of the control handle. A tip or tip part may be positioned at the distal end of the elongated insertion tube. The tip may further comprise a bending section positioned between the tip and the elongated insertion tube. The bending section may be configured to be articulated to maneuver the endoscope inside a body cavity.

The bending section may be bendable in one or two dimensions, e.g. an up/down dimension and a left/right dimension. The bendable tip may comprise a bending section with increased flexibility, e.g. achieved by articulated segments of the bending section as are known in the art. The steering wire(s) may run along the inside of an elongated insertion tube from the tip through the bending section to the control system positioned in or forming part of the endoscope handle.

The endoscope may be a disposable insertion endoscope, which is intended to be used and discarded and not sterilized for a subsequent use.

A third aspect of the present disclosure relates to a video processing system comprising an endoscope according to the second aspect and a video processing apparatus.

A person skilled in the art will appreciate that any one or more of the above aspects of this disclosure and embodiments thereof may be combined with any one or more of the other aspects and embodiments thereof.

### BRIEF DESCRIPTION OF THE DRAWINGS

In the following, non-limiting exemplary embodiments will be described in greater detail with reference to the drawings, in which:

FIG. 1 shows a side view of an embodiment of an endoscope including an embodiment of a control handle;

FIG. 2 shows a side view of a portion of the endoscope of FIG. 1;

FIG. 3 shows a perspective view of the endoscope of FIG. 2;

FIG. 4 shows a palm-side view of the endoscope of FIG. 2;

FIG. 5 shows an exploded perspective view of the of the endoscope of FIG. 2;

FIGS. 6-14 are views of cross-sections of the handle housing of the control handle of FIG. 2;

FIG. 15 shows a perspective view of another embodiment of an endoscope;

FIG. 16 shows a finger-side view of the embodiment of the endoscope of FIG. 15; and

FIG. 17 shows an exploded perspective view of a variation of the embodiment of the endoscope of FIGS. 15 and 16;

FIG. 18 shows a perspective view of the embodiment of the endoscope of FIG. 17;

FIG. 19 depicts an embodiment of a video processing apparatus with an integrated display screen;

FIG. 20 depicts an embodiment of a video processing apparatus without an integrated display screen;

FIG. 21 is a block diagram of a video processing system including a video processing apparatus as in FIG. 19 or 20; and

FIG. 22 is a block diagram of components of the video processing apparatus of FIGS. 19 and 20.

### DETAILED DESCRIPTION

Herein, the term “patient” includes humans and animals.

Herein, the hand of the operator is described as having four fingers (index, middle, ring and little fingers) and a thumb. The pronoun “their” is used to identify the operator in a gender-neutral manner.

Herein, the terms “proximal” and “distal” are used to describe position in relation to a longitudinal axis of the endoscope and proximity to an operator of the endoscope. Accordingly, the longitudinal axis defines a proximal-distal direction, whereby when the handle is held by the operator the handle is proximal to the operator and the camera is distal of the operator. Transversely to the proximal-distal direction, the terms “right” and “left” are used to describe sides of the handle orthogonally to the “palm side” and “finger side” of the handle. The palm side is the side of the handle on which the palm of the operator rests, and the finger side is the side about which the fingers curve to grip the endoscope.

As mentioned, endoscopes encompass a broad category of devices operable to obtain images depicting internal tissues and structures of a patient. The handle includes a bending mechanism operable to articulate the bending section and thereby orient the field of view of the camera along a palm/finger plane or along a right/left plane.

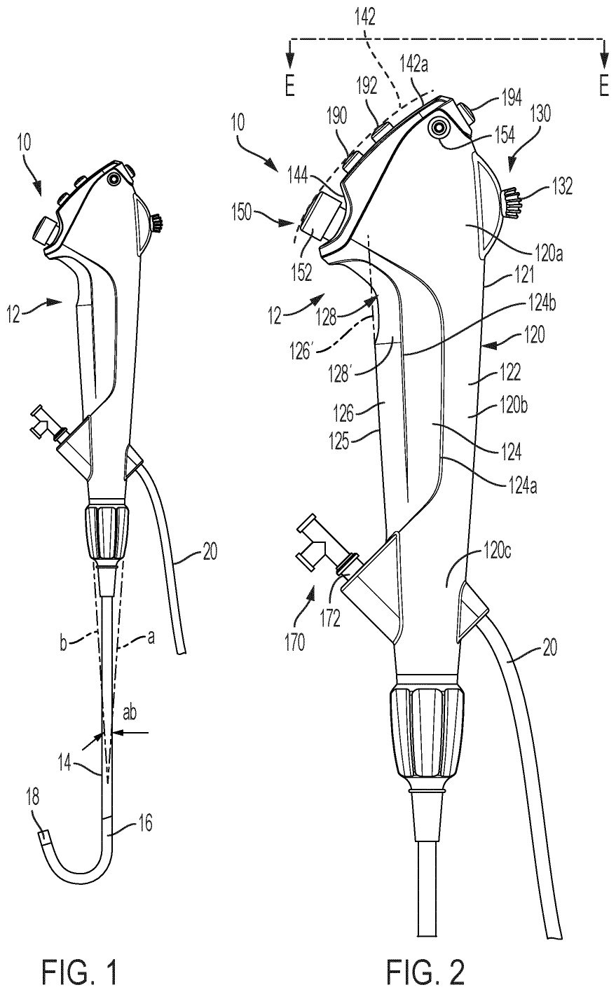

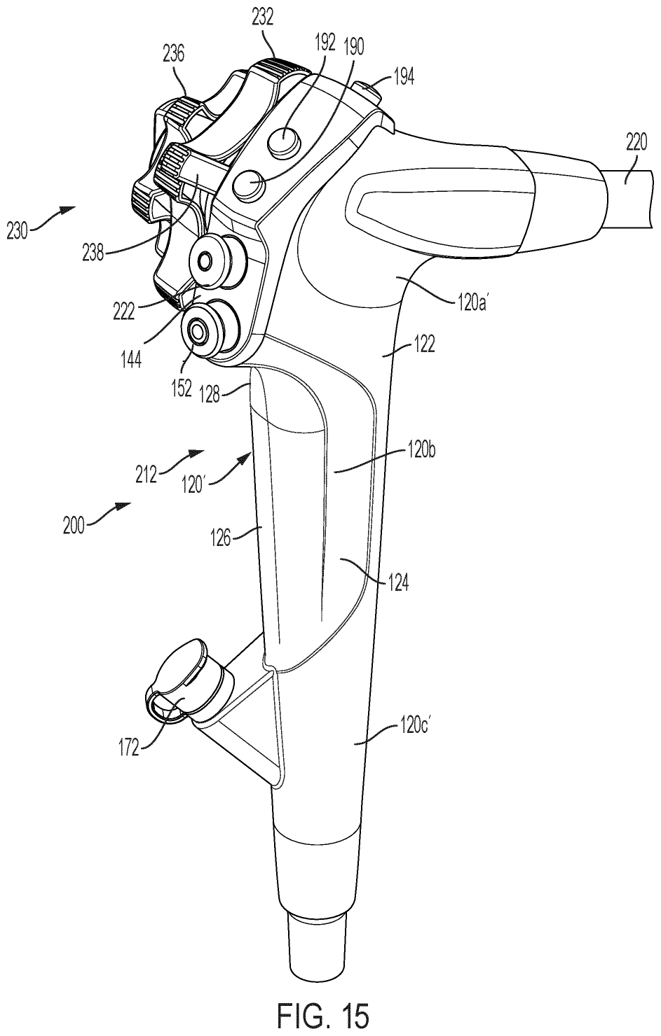

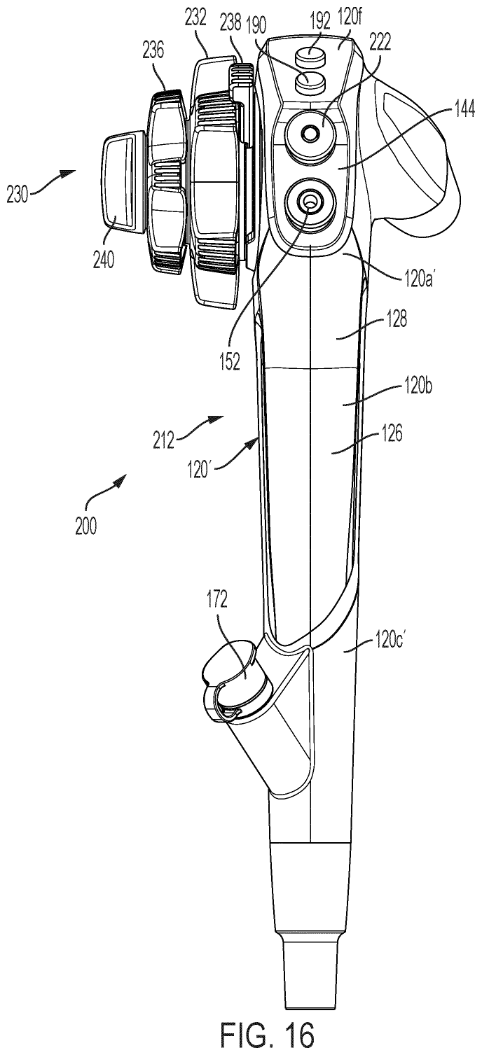

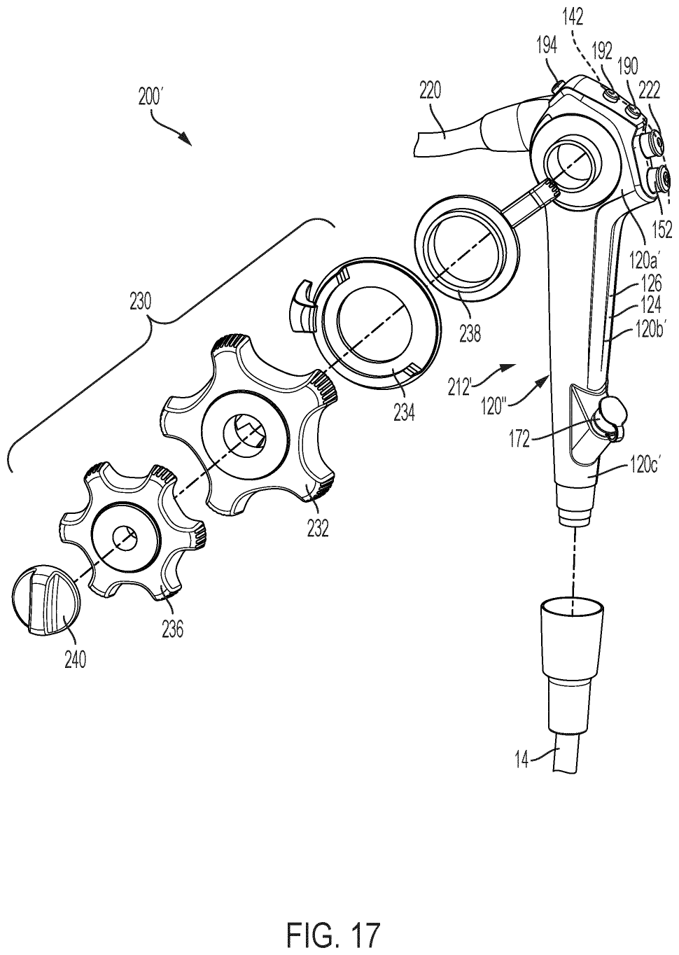

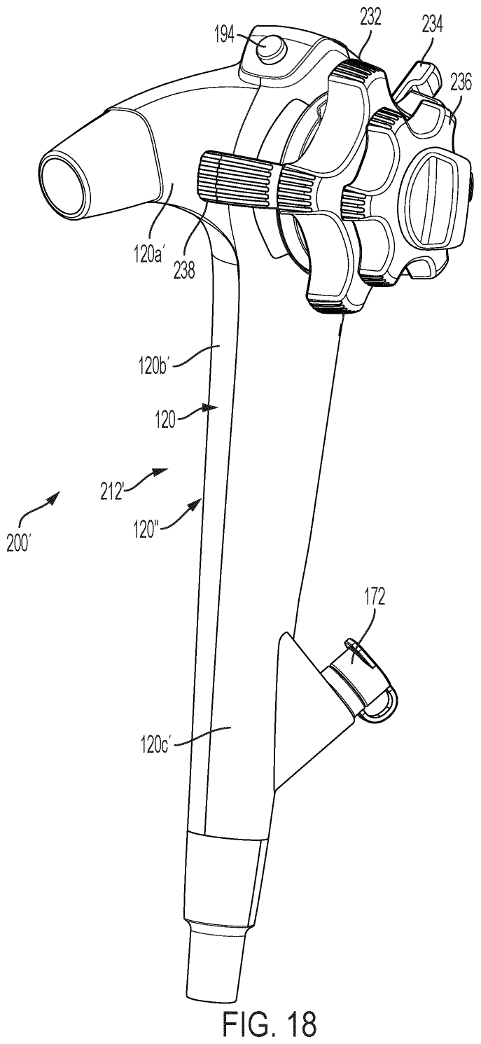

FIGS. 1-14 depict an embodiment of an endoscope 10 including a control handle 12 comprising a handle housing 120 having a grip section 120b intermediate a proximal end 120a and a distal end 120c. FIGS. 15 and 16 depict another embodiment of an endoscope, denoted by numeral 200, including a control handle 212 comprising a handle housing 120′ having grip section 120b intermediate a proximal end 120a′ and a distal end 120c′. Endoscope 10 has a one-dimensional steering mechanism and endoscope 200 has a two-dimensional steering mechanism. As shown, the endoscopes have the same grip section, albeit the dimensions may vary based on the features of various implementations of each embodiment of the endoscope. FIGS. 17 and 18 depict a variation of the embodiment of endoscope 200, denoted by numeral 200′. Endoscope 200′ includes a control handle 212′ comprising a handle housing 120″ having grip section 120b′ intermediate proximal end 120a′ and distal end 120c′. Endoscope 200′ differs from endoscope 200 in that the grip section, denoted by numeral 120b′, does not include undercut 128. In all other respects endoscope 200′ is the same as endoscope 200. Similarly, in a variation of endoscope 10, endoscope 10 could omit undercut 128.

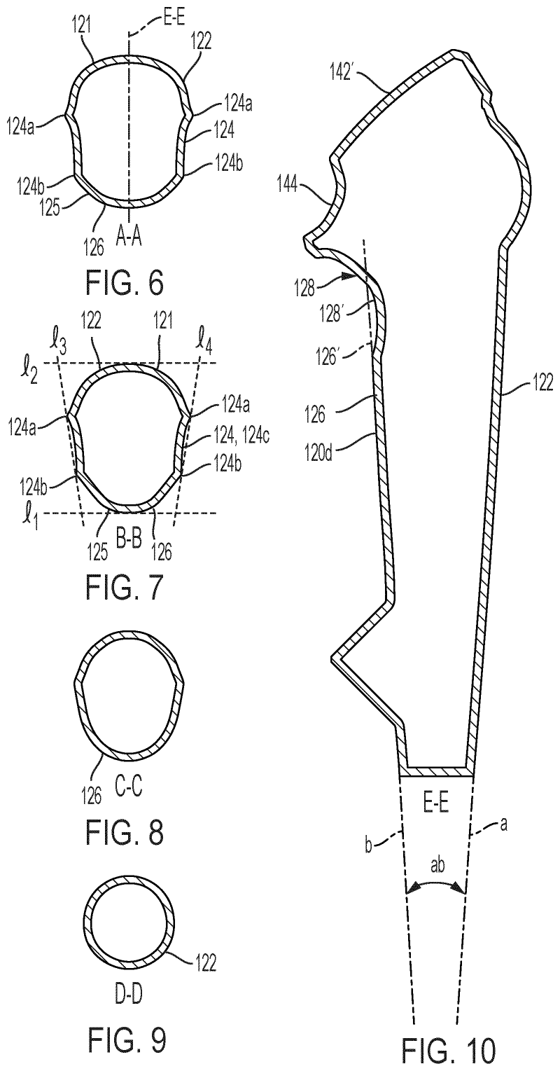

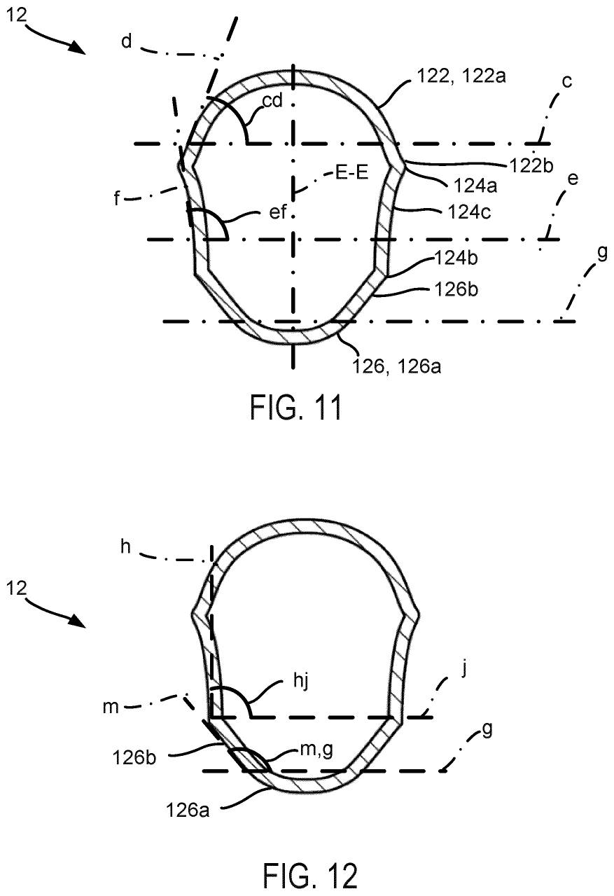

Referring to FIGS. 1 and 2, endoscope 10 comprises control handle 12 comprising handle housing 120 having proximal end 120a, distal end 120c longitudinally opposite the proximal end, a palm side 121, and a finger side 125 transversely opposite the palm side, and grip section 120b between the proximal end and the distal end. The grip section has a finger undercut 128, a palm surface 122 on the palm side, a finger surface 126, 128′ on the finger side, opposite the palm surface, and elongate longitudinal recesses 124 intermediate the finger surface and the palm surface, the palm surface and the finger surface being transversely convex. Finger surface 128′ is a surface of finger undercut 128. Elongate recesses 124 may extend between longitudinal lines or ridges defined by the juncture of finger surface 126 and elongate recesses 124, denoted as ridges 124b (best seen in FIG. 6), and by the juncture of palm surface 122 and elongate recesses 124, denoted as ridges 124a (best seen in FIG. 6). Elongate recesses 124 may be, in the transverse direction along the grip section, concave, convex, or substantially flat. If concave, the longitudinal lines are inflection lines reflecting a discontinuous change in the curvature. If convex, the curvature radius thereof is larger than a curvature radius of the grip surface. The largest radius of the palm surface may be smaller than the smallest radius of the elongate recesses' surfaces measured on the same transverse plane along the grip section, such that the elongate recess surfaces are “flatter” transversely than the palm and grip side surfaces 122, 126. By discontinuous it is meant that, on a common plane traversing two surfaces, an angle between the tangents of the two surfaces at points on the two surfaces closest to each other is greater than 20 degrees, preferably more than 30 degrees. This is further discussed with reference to FIG. 11. The two surfaces may contact at an edge. An intermediate surface having a small radius of curvature may be provided instead of an edge.

In use, a user of the endoscopes described herein has to reach a number of controls, described below, with their thumb, index and middle fingers, which requires gripping of the handle with the ring and little fingers, referred to herein as the “handle gripping fingers” as opposed to the “control fingers” which include the thumb and index and middle fingers. Of course, this definition does not preclude that a user at times grip the handle also with the thumb and middle or index fingers. Recesses 124 provide a location for the pad of the tip of each of the gripping fingers. Because the finger pads have a high number of nerves, the fingertips are sensitive and the structure of the handle gives the user a good and secure gripping sense. Because of the sensitivity of the fingertips the user immediately knows how the hand is positioned on the handle with respect to other interaction points such as the bending control lever, where the thumb is positioned, and suction and control buttons typically activated with the index and middle fingers. The finger surface is smaller than the palm surface to accommodate the curvature of the gripping fingers. The ridges may provide tactile feedback for positioning the creases of the gripping fingers thereon. Thus, the palm of the user rests on the palm surface, the gripping fingers wrap around the finger surfaces, and the fingertips of the gripping fingers rest on one of the elongate recesses. The recesses form a natural rest for the fingertip pads. Moreover, during medical procedures it may be required to have a firm grip on the handle to perform a torsional rotation of the handle and the insertion tube to maneuver the distal tip. For that purpose, fingertip pads may be positioned in the recess to provide a solid grip on the handle. The handle could be made with an elongate recess on one side only, if the handle were designated as a right hand or left hand only handle but providing both elongate recesses allows ambidextrous use.

The palm surface and/or the finger surface may be textured to improve grip. The texture may be stippled in any pattern including a random dot pattern and lined patterns.

An insertion tube 14 is connected to and extends distally from distal end 120c of handle housing 120. A bending section 16 is connected to and extends distally from insertion tube 14. A camera 18 is connected to and extends distally from bending section 16. A cable 20 includes wires connected to camera 18 to provide power to an image sensor and a light source therein, and to transfer image data from the image sensor through cable 20 to a video processing device. The video processing device may also provide the power for the camera. Additional wires may be provided to transfer control signals from control handle 12 to the video processing device. Bending section 16, insertion tube 14, camera 18, and cable 20 are also provided in various embodiments disclosed below including embodiments of endoscopes 200 and 200′.

Grip section 120b may be symmetric about a longitudinal palm/finger plane E-E extending therethrough. Palm surface 122 defines a straight line “a” on plane E-E and finger surface 126 defines a straight line “b” on plane E-E. The straight lines, a and b, form an angle “ab” with a vertex distal of the distal end of the handle housing. The “V” shape of the handle housing provides a larger internal volume at the proximal end than at the grip section, which advantageously provides a comfortable and ergonomic grip section while providing sufficient space for incorporation of various control features described below. In other embodiments lines a and be could be slightly or substantially curved. The transverse distance on plane E-E at the distal end of the handle is smaller than the transverse distance on plane E-E at grip section 120b.

Handle housing 120 may be provided as two elongate halves (shown in FIG. 4 as 120d, 120e) and, optionally, a proximal portion (shown in FIG. 4 as 120f) enclosing an internal volume of handle housing 120. The proximal portion can also be part of the two elongate halves.

An optional undercut surface 128′ may be connected to and extend proximally from finger surface 126. Undercut surface 128′ is curved along the longitudinal plane and curves toward palm surface 122, a portion of undercut surface 128′ being positioned between a longitudinal projection 126′ of line b and palm surface 122, before curving in the opposite direction as it extends toward the proximal end. Undercut 128 allows the operator to grip the handle more proximally and comfortably, by reducing the transverse distance between lines a and b at the proximal end of the grip section, where the transverse distance would otherwise be largest, while providing a positive index for the index or middle fingers, which enables the operator to position their hand properly on the handle without looking at it. Undercut 128 can be omitted, as shown in FIGS. 17 and 18.

Although typically the user will hold the handle with the finger pads in the elongate recess 124, a reverse grip is also possible. In the reverse grip, the user places the palm in the finger surface and the fingers wrap around the palm surface, with the index finger controlling control lever 132. A pistol grip is also possible. In the pistol grip, the user places the index finger in finger undercut 128 and holds the portion of the handle proximal of undercut 128 while placing the thumb on control lever 132. Accordingly, the features disclosed herein improve the user's traditional grip and also alternative ways to grip the endoscope.

The recess and the ridge positioned closest to the palm surface also serve as guidance to obtain access to the working channel with the hand opposite the gripping hand. When gripping the handle with one hand and with at least one finger positioned in the recess, the user may even without looking at the handle easily locate a finger positioned in the recess and follow the recess and the ridge in the distal direction in order to locate the position of the working channel port. Again, the features of the handle disclosed herein facilitate use of the endoscope by a user without necessarily viewing the endoscope handle to determine proper hand placement and operation of control buttons, suction port etc.

Recesses 124 extend on opposite sides of the handle and are closer to each other than longitudinal edges of the palm surface, thus being recessed relative to projections of the palm surface extending toward the finger surface. The recesses thus provide a reduced width to the grip section than the palm section encompassing the palm surface.

The control handle also comprises a bending mechanism 130 including a bending control lever 132, a suction mechanism 150 including a suction button 152 and a suction port 154, a working channel arrangement 170 comprising a working channel port 172, and control buttons 190, 192, and 194.

A working or suction channel may run along the inside of the insertion tube from the handle to the tip part, e.g. allowing liquid to be removed from the body cavity or allowing for insertion of surgical instruments or the like into the body cavity. The suction channel may be connected to suction port 154 and operated with suction button 152 in a conventional manner.

Control buttons 190, 192, and 194 can be actuated by the user to implement various control features. The control features may be preset or programmable. For example, the control buttons can communicate control signals to the video processing apparatus to invert the image captured by the camera, to change the intensity of the image (brighter/darker), to save an image, etc. The control buttons may activate electric switches connected via wires passing through cable 20 to the video processing apparatus. The video processing apparatus may, in turn, communicate corresponding signals to the image sensor. The processing apparatus may perform various functions on the control signals to generate the corresponding signals, such as converting them from on/off signals to a communication protocol used by the image sensor, such as an I₂C protocol which is well known and commonly used.

Advantageously, the shape of the grip section and the handle enable the operator to comfortably, with one hand, grip the handle, rotate bending control lever 132, operate suction button 152, and actuate control buttons 190, 192, and 194. As shown in FIG. 2, a dashed line 142 drawn as a continuous curve (i.e. without discontinuities) can be drawn over the top surfaces of suction button 152 and control buttons 190 and 192. Line 142 has the curvature of a proximal surface 142a of handle 120 due to control buttons 190 and 192 having the same height. A recess 144 is provided adjacent surface 142a, where suction button 152 is positioned. Because suction button 152 is larger than control buttons 190 and 192, recess 144 permits the top surface of suction button 152 to be placed along line 142. In a more general example, the proximal surface of the handle is curved, as is line 142, and the top surfaces of all the buttons, regardless of their sizes, are aligned with line 142, which may be referred to as the reach line. The proximal wall comprising surface 142a may have one or more recesses, depending on the sizes of the buttons, to allow the top surfaces to align with the reach line. The reach line is designed so that the user, with their thumb on control lever 132, can move the tip of their index finger from one to the other buttons with the same amount of extension, which is comfortable for the user. Recess 144 may be comprise an arc, a saddle shape, or any other shape that permits activation of the button while allowing its top surface to follow the reach line. The curvature of the reach line, and its distance from the palm surface and recess 128, may be determined statistically to fit a group of users, such as a group of users with average sized hands or hands smaller than average, for example. Different endoscope handle sizes may also be provided and the characteristics of the reach line may be designed to correspond to handle/hand sizes.

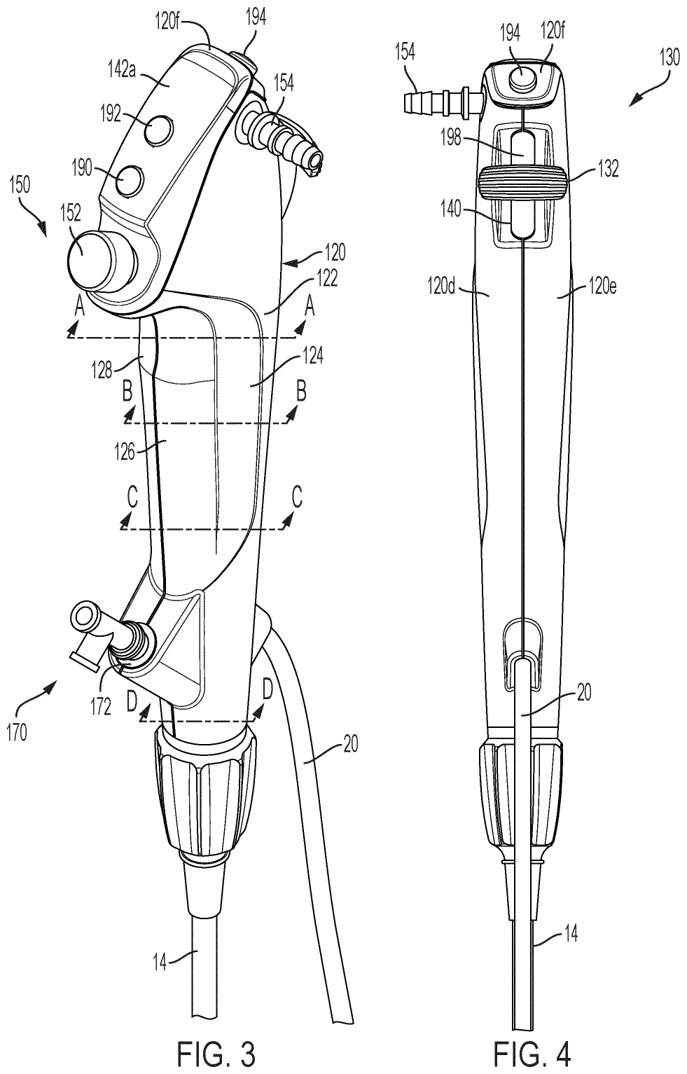

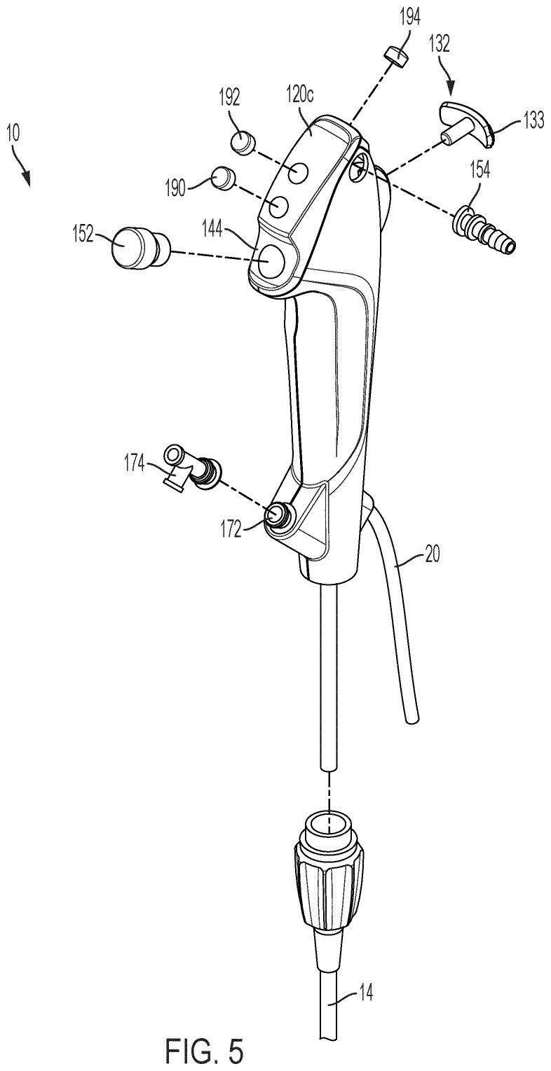

FIGS. 3 and 4 provide perspective and palm-side views of endoscope 10. Referring to FIG. 3, transverse planes A-A, B-B, C-C, and D-D are shown and are described with reference to FIGS. 5-8. Referring to FIG. 4, bending mechanism 130 includes a roller having a surface 198 visible through a slot 140 in handle housing 120. Control lever 132 can rotate along slot 140 to rotate the roller, and the roller thus translates control wires that cause the bending section to bend, in a conventional manner.

FIGS. 6 to 9 illustrate that the periphery of the palm surface at each cross-section decreases in length progressively from cross-section A-A, B-B, C-C, and D-D, as these cross-sections approach the vertex of angle ab. These cross-sections also illustrate the convex shapes of the palm side and the finger side, and the substantially flat surface, in the transverse direction, of recesses 124. FIGS. 6 and 7 clearly show ridges 124a at the juncture of palm surface 122 and the surfaces of recesses 124. Elongate recesses 124 extend between ridges 124a and 124b. Longitudinal sides of palm surface 122 may extend outwardly slightly, e.g. the palm surface in a transverse plane being convex but having concave ends with substantially smaller curvature radius, to make ridges 124a extend outwardly and thus be easier to feel. Ridges 124b may be formed by the juncture of the finger surface 126 and the concave recess surface, always in the transverse plane. Of course, a small concave curvature may be added to the finger surface to accentuate ridges 124b in the same manner as in ridges 124a.

FIG. 7 shows lines l1, l2, l3, and l4. Lines l1 and l2 are parallel and tangentially positioned adjacent the palm and finger surfaces. Lines l3 and l4 are positioned tangentially to ridges 124a and 124b. The lines illustrate that finger side 125, in a transverse direction, is smaller than palm side 121. This gives the user tactile feedback regarding the orientation of the handle. Lines (3 and (4 also illustrate the concave nature of the elongate recess surfaces 124c.

FIG. 10 illustrates the shape of one-half of the handle housing, in particularly the concave shape of finger undercut 128 and the orientation of the curvature of the finger undercut relative to projection 126′, as discussed above.

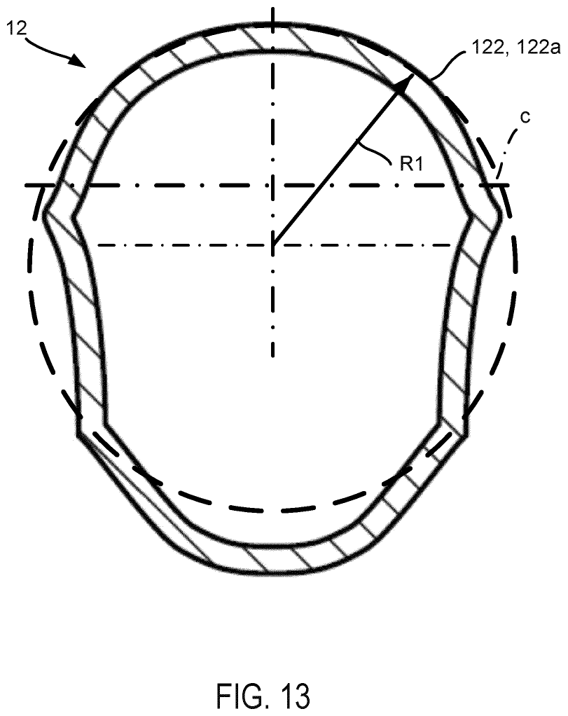

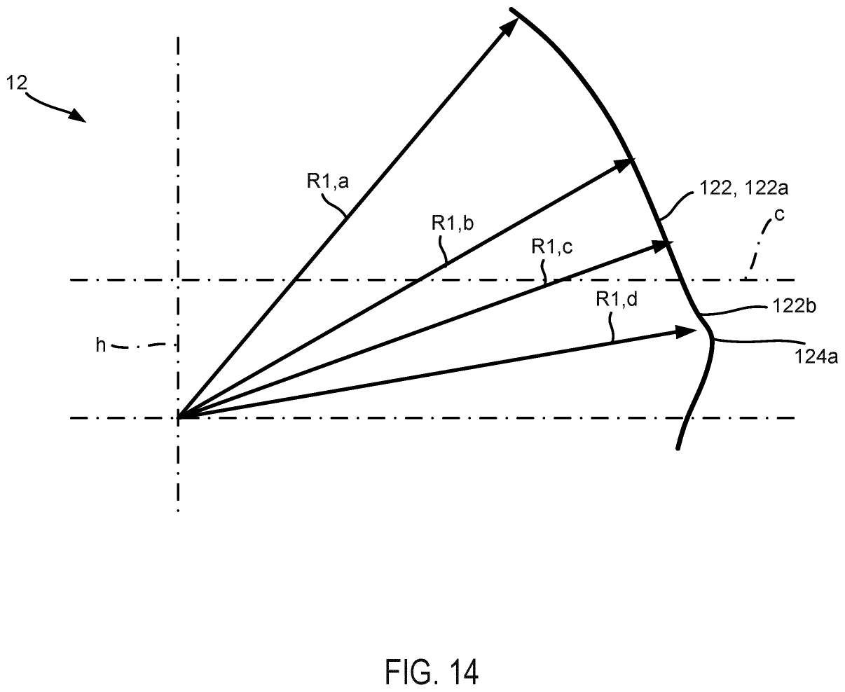

FIGS. 11 to 14 expand on the geometry of the cross-sections of the handle described with reference to FIGS. 6 to 9. Therein are shown lines c, d, e, f, g, h, j, and m, which are orthogonal to plane E-E, and angles cd, ef, hj, and mg. Line c is positioned at the inflection points between the convex and concave portions of palm surface 122, illustrating a convex portion 122a and, extending therefrom, portions 122b. Angle cd is the angle of a line tangential to convex portion 122a at the inflection point. Angle ef is the smallest angle of a line f tangential to elongate recess surface 124c. The difference between angles cd and ef is at least 20 degrees, indicating a discontinuity in the curvature of the surface of the handle. The difference is represented by an angle formed by lines f and d. Preferably the difference is at least 30 degrees. It should be understood that the differences are in the context of the left side of FIG. 11 and thus, more broadly, the absolute values of the differences are intended to depict discontinuities in the surfaces. Additionally, it is noted that as shown in FIG. 11, the entirety of elongate recess surface 124c, in a traverse plane, extends toward plane E-E between ridges 124a and 124b and is thus concave. The same geometry is evident with respect to finger surface 126, where line g is positioned at the inflection points between the convex and concave portions of finger surface 126, illustrating a convex portion 126a and, extending therefrom, portions 126b. Portions 122b and 126b can be straight or concave. FIG. 13 depicts a circle with a radius R1 encompassing most of portion 122a of palm surface 122. The curvature radius then gradually decreases until the inflection points illustrated by line c. FIG. 14 illustrates the gradual decrease by showing radius R1, a-d, to illustrate how the radius (e.g. the tips of the lines) at positions a-d touch (at position a) or extends past (at positions b, c) or do not reach (at position d) palm surface 122. On variations of the present embodiment the curvature radius remains constant until the inflection points.

FIGS. 15 to 18 depict endoscopes 200, 200′ comprising a two-dimensional steering mechanism 230 including a first control wheel 232, a first brake wheel 234, a second control wheel 236, a second brake wheel 238, and a knob 240 provided to secure the wheels concentrically onto a handle 212. Any known two-dimensional steering mechanism may be used. First and second control wheels 232 and 236 comprise spools or arcuate portions operable to translate control wires in a known manner and thus articulate the bending section in the palm/finger plane or the right/left plane. As stated previously, endoscope 200′ differs from endoscope 200 in that grip section 120b′ does not include undercut 128. Endoscopes 200, 200′ also comprise a button 222 positioned adjacent button 152 and a cable 220 which is functionally equivalent to cable 20. The top surfaces of buttons 190, 192, 152, and 222 follow the reach line (line 142, shown in FIG. 13) as with endoscopes 10 and 200′.

Handle housing 212 has a grip section 120b intermediate a proximal end 120a′ and a distal end 120c′. Distal end 120c′ is substantially the same as distal end 120c and proximal end 120a′ differs from proximal end 120a in that the two-dimensional endoscope requires more space at the proximal end of the handle to locate the control wire control mechanism and the brake wheels. Referring to FIGS. 13 and 14, handle 212′ has handle housing 120″ with a grip section 120b′ intermediate proximal end 120a′ and distal end 120c′. Grip section 120b′ differs from grip section 120b in that it is devoid of finger undercut.

The endoscopes described herein are assembled by positioning the various control components in the handle and then securing the opposite handle parts to each other.

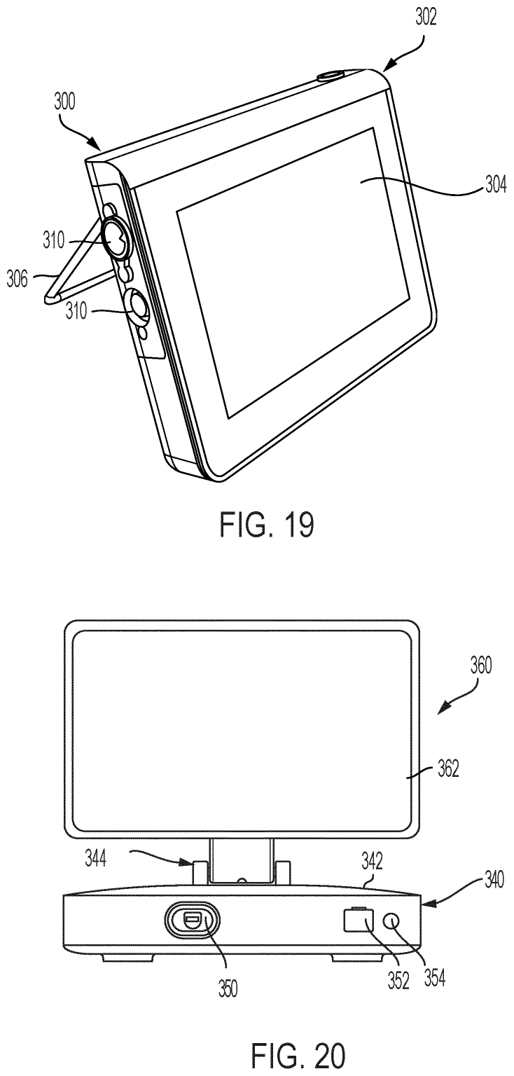

FIG. 19 depicts an embodiment of a video processing apparatus, or medical monitor, 300 having an integrated display screen 304 supported by a housing 302. A pivotable stand 306 is shown supporting video processing apparatus 300 on a support surface (not shown). Connector ports 310 are shown. Connectors of endoscopes can be plugged into connector ports 310 to establish communications with the endoscopes and to provide power to them. Display screen 304 may be a touch sensitive screen that also functions as a user input sensor.

FIG. 20 depicts an embodiment of a video processing apparatus 340 comprising a housing 342, a connector port 310, a power button 352, and an indicator light 354. More than one connector port 310 may be provided, and connector ports 310 may be positioned in the rear side of video processing apparatus 340. A display support 344 is provided so that a display 360 having a display screen 362 may be mounted thereon. Display support 344 is optional. Instead, display 360 can be wall or IV mounted and connected wirelessly or via a cable to video processing apparatus 340 or video processing apparatus 300.

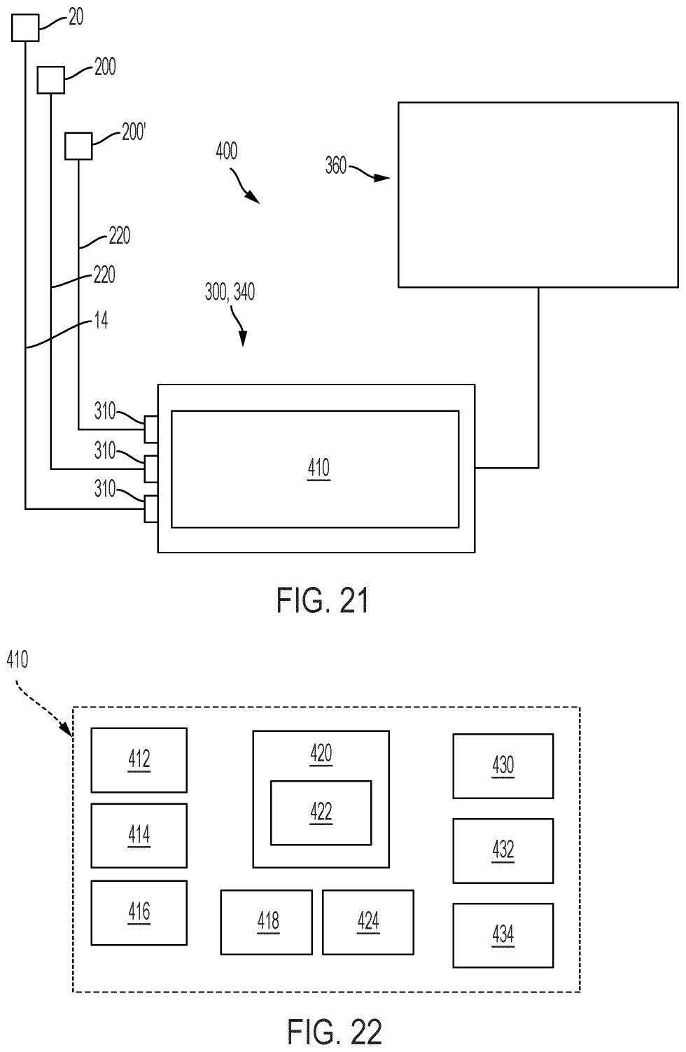

FIG. 21 is a block diagram of a video processing system 400 including a video processing apparatus as in FIGS. 19 and 20. FIG. 21 illustrates connection of endoscopes 20, 200, and 200′ via cables 14 and 220, to connector ports 310. The connector ports 310 enable concurrent transfer of images from the endoscopes. By concurrent it is meant that one, two or three live images or video streams from the endoscopes may be presented on a display screen at practically the same time. Multiplexing may be used to present the images and such presentation, as used herein, is concurrent. A is shown, including various components described with reference to FIG. 22.

FIG. 22 is a block diagram of motherboard 410. Motherboard 410 has connected to it medical device interfaces 412, 414, and 416 operable to connect to each endoscope. The interfaces may comprise different logic depending on which endoscope will be connected to each interface so that they are technically compatible. A processor 418, a memory 420 including graphical user interface (GUI) logic 422, a field-programmable gate array (FPGA) 424, video output board 430, a user interface 432, and a microphone 434 are also provided. User interface 430 may comprise a wireless interface operable to receive user inputs via a mouse, keyboard, or other physical user input devices. Example wireless interfaces include Bluetooth and Zigbee controllers. User interface 430 may also comprise a USB port to receive a USB connector including the wireless interface or a USB connector of a wired user input device.

FPGA 424 is optionally provided because it is capable of rapid power-up (i.e. short boot-up time) and thus is useful in emergency situations. FPGAs may also be provided in the medical device interfaces for the same reasons. FPGAs process data very fast compared to other memory/instruction combinations and are re-programmable. Therefore, FPGAs facilitate presentation of a live view of the images captured by the videoscope in real-time with minimal latency so that the physician observing the live view can take immediate actions even in emergency situations. As technology evolves, the functionality of FPGA 424 may be combined with processor 418. Processor 418 may perform any function, such as operating fans and communications via user interface 432.

While the invention has been described as having exemplary designs, this application is intended to cover any variations, uses, or adaptations of the disclosed examples and embodiments in accord with the appended claims.

### LIST OF REFERENCE NUMERALS

10 endoscope
12 handle
212 handle
14 insertion tube
16 bending section
18 camera
20 cable
200 endoscope
200′ endoscope
120 handle housing
120′ handle housing
120a proximal end
120a′ proximal end
120b grip section
120b′ grip section
120c distal end
120c′ distal end
120d housing half
120e housing half
120f housing proximal portion
121 palm side
122 palm surface
124 longitudinal recess
125 finger side
126 finger surface
128 finger undercut
128′ finger surface
130 bending mechanism
132 bending control lever
133 articulation knob
134 roller
136 bending control wire
140 slot
150 suction mechanism
152 suction button
154 suction port
156 suction aperture
158 suction tube
170 working channel arrangement
172 working channel port
190 control button
182 control button
194 control button
196 axle cavity
197 bending control cable wheel
198 roller surface
230 two-dimensional steering mechanism
232 first control wheel
234 first brake wheel
236 second control wheel
238 second brake wheel
240 knob

We claim:

1. A control handle for an endoscope, the control handle comprising:
a handle housing having a proximal end, a distal end longitudinally opposite the proximal end, and a grip section between the proximal end and the distal end, the grip section having a palm surface, a finger surface opposite the palm surface, and elongate longitudinal recesses intermediate the finger surface and the palm surface, the palm surface having a convex portion in a transverse plane, the finger surface having a convex portion in the transverse plane, and each of the elongate longitudinal recesses having an elongate recess surface between the palm surface and the finger surface, each elongate recess surface having a concave portion in the transverse plane.

2. An endoscope comprising a control handle, the control handle comprising:
a handle housing having a proximal end, a distal end longitudinally opposite the proximal end, and a grip section between the proximal end and the distal end, the grip section having a palm surface, a finger surface opposite the palm surface, and elongate longitudinal recesses intermediate the finger surface and the palm surface, the palm surface having a convex portion in a transverse plane, the finger surface having a convex portion in the transverse plane, and each of the elongate longitudinal recesses having an elongate recess surface between the palm surface and the finger surface, each elongate recess surface having a concave portion in the transverse plane,
wherein the handle housing comprises elongate ridges between the finger surface and the elongate recess surfaces and between the elongate recess surfaces and the palm surface, the elongate recess surfaces extending between the ridges on either side of the handle housing without crossing planes tangential to the ridges, and
wherein the grip section is symmetric about a longitudinal plane extending therethrough, wherein the finger surface includes a longitudinal convex surface and an undercut surface connected to and extending proximally from the longitudinal convex surface, wherein the undercut surface is curved along the longitudinal plane and curves toward the palm surface, a portion of the undercut surface positioned between a longitudinal projection of the longitudinal convex surface and the palm surface.

3. The control handle of claim 1, wherein the handle housing comprises elongate ridges between the finger surface and the elongate recess surfaces and between the elongate recess surfaces and the palm surface.

4. The control handle of claim 3, wherein the palm surface includes concave portions extending from both ends of the convex portion to the elongate ridges.

5. The control handle of claim 4, wherein the elongate recess surfaces extend between the ridges on either side of the handle housing without crossing planes tangential to the ridges.

6. The control handle of claim 3, wherein the grip section is symmetric about a longitudinal plane extending therethrough, wherein the palm surface defines a straight line “a” on the longitudinal plane, wherein the finger surface defines a straight line “b” on the longitudinal plane, and wherein the straight lines “a” and “b” form an angle with a vertex distal of the distal end of the handle housing.

7. An endoscope comprising a control handle, the control handle comprising:
a handle housing having a proximal end, a distal end longitudinally opposite the proximal end, and a grip section between the proximal end and the distal end, the grip section having a palm surface, a finger surface opposite the palm surface, and elongate longitudinal recesses intermediate the finger surface and the palm surface, the palm surface having a convex portion in a transverse plane, the finger surface having a convex portion in the transverse plane, and each of the elongate longitudinal recesses having an elongate recess surface between the palm surface and the finger surface, each elongate recess surface having a concave portion in the transverse plane,
wherein the handle housing comprises a palm side, a finger side opposite the palm side, a proximal surface between the palm side and the finger side, a first control button, a second control button, and a third control button,
wherein the third control button is larger than the first control button,
wherein the proximal surface comprises a first portion that is curved and a second portion forming a recess that is distal of the first portion,
wherein the first control button and the second control button extend proximally from the first portion and the third control button extends proximally from the second portion, and
wherein proximal surfaces of the first control button, the second control button and the third control button lie along a reach line having a first portion parallel to the first portion of the proximal surface and a second portion extending a curvature of the first portion.

8. The control handle of claim 1, wherein the palm surface and/or the finger surface are textured.

9. An endoscope comprising the endoscope handle of claim 1.

10. The endoscope of claim 9, further comprising a cord including an insertion tube, a bending section, a tip housing and a camera positioned in the tip housing.

11. A video processing system comprising:
the endoscope of claim 9, the endoscope including a cable with a connector; and
a video processing apparatus comprising a video interface operable to communicatively couple with the connector of the endoscope.

12. The control handle of claim 1, wherein the grip section is symmetric about a longitudinal plane extending therethrough, wherein the finger surface includes a longitudinal convex surface and an undercut surface connected to and extending proximally from the longitudinal convex surface, wherein the undercut surface is curved along the longitudinal plane and curves toward the palm surface, a portion of the undercut surface positioned between a longitudinal projection of the longitudinal convex surface and the palm surface.

13. The control handle of claim 1, wherein the handle housing comprises a palm side, a finger side opposite the palm side, a proximal surface between the palm side and the finger side, a first control button, a second control button, and a third control button,
wherein the third control button is larger than the first control button,
wherein the proximal surface comprises a first portion that is curved and a second portion forming a recess that is distal of the first portion,
wherein the first control button and the second control button extend proximally from the first portion and the third control button extends proximally from the second portion, and
wherein proximal surfaces of the first control button, the second control button and the third control button lie along a reach line having a first portion parallel to the first portion of the proximal surface and a second portion extending a curvature of the first portion.

14. The control handle of claim 1,
wherein the handle housing comprises elongate ridges between the finger surface and the elongate recess surfaces and between the elongate recess surfaces and the palm surface,
wherein the handle housing comprises a palm side, a finger side opposite the palm side, a proximal surface between the palm side and the finger side, a first control button, a second control button, and a third control button,
wherein the third control button is larger than the first control button,
wherein the proximal surface comprises a first portion that is curved and a second portion forming a recess that is distal of the first portion,
wherein the first control button and the second control button extend proximally from the first portion and the third control button extends proximally from the second portion, and
wherein proximal surfaces of the first control button, the second control button and the third control button lie along a reach line having a first portion parallel to the first portion of the proximal surface and a second portion extending a curvature of the first portion.

15. The control handle of claim 1, wherein the grip section is symmetric about a longitudinal plane extending therethrough, wherein the finger surface includes a longitudinal convex surface and an undercut surface connected to and extending proximally from the longitudinal convex surface, wherein the undercut surface is curved along the longitudinal plane and curves toward the palm surface, a portion of the undercut surface positioned between a longitudinal projection of the longitudinal convex surface and the palm surface.

16. A video processing system comprising:
the endoscope of claim 2, the endoscope further comprising an insertion cord, the insertion cord including an insertion tube, a bending section, a tip housing, and a camera in the tip housing; and
a video processing apparatus comprising a video interface operable to communicatively couple with the endoscope.

17. A video processing system comprising:
the endoscope of claim 7, the endoscope further comprising an insertion cord, the insertion cord including an insertion tube, a bending section, a tip housing, and a camera in the tip housing; and
a video processing apparatus comprising a video interface operable to communicatively couple with the endoscope.
